(19) United States
(12) Patent Application Publication
(10) Pub. No.: US 2022/0202869 A1
(43) Pub. Date: Jun. 30, 2022

(54) MESENCHYMAL STEM CELLS FOR TREATMENT OF SKIN DISORDERS AND SKIN PROBLEMS

(71) Applicant: Riam Shammaa, Toronto (CA)

(72) Inventor: Riam Shammaa, Toronto (CA)

(21) Appl. No.: 17/527,148

(22) Filed: Nov. 15, 2021

Related U.S. Application Data
(60) Provisional application No. 63/130,501, filed on Dec. 24, 2020.

(51) Int. Cl. A61K 35/28; A61P 17/02

(52) U.S. Cl. CPC A61K 35/28; A61P 17/02

(57) ABSTRACT

A composition for treatment of skin disorders and skin problems comprising mesenchymal stem cells wherein said mesenchymal stem cells express CD146 receptors in an amount of at least 10 pg/10⁶ cells.

### CROSS-REFERENCE TO RELATED APPLICATION

This application claims the benefit under 35 U.S.C. § 119 of U.S. Provisional Patent Application No. 63/130,501, filed on Dec. 24, 2020, which is incorporated herein by reference in its entirety.

### FIELD OF THE INVENTION

The present invention relates to mesenchymal stem cells (MSCs), and in particular, to MSCs for treatment of skin disorders and skin problems.

### BACKGROUND OF THE INVENTION

MSCs are multipotent stem cells that can differentiate readily into lineages including osteoblasts, myocytes, chondrocytes, and adipocytes (Pittenger, et al., Science. Vol. 284, pg. 143 (1999); Haynesworth, et al., Bone. Vol. 13, pg. 69 (1992); Prockop, Science, Vol. 276, pg. 71 (1997)). In vitro studies have demonstrated the capability of MSCs to differentiate into muscle (Wakitani, et al., Muscle Nerve. Vol. 18, pg. 1417 (1995)), neuronal-like precursors (Woodbury, et al., J. Neurosci. Res., Vol. 69, pg. 908 (2002); Sanchez-Ramos, et al., Exp. Neurol., Vol. 171, pg. 109 (2001)), cardiomyocytes (Toma, et al., Circulation. Vol. 105, pg. 93 (2002); Fukuda, Artif. Organs. Vol. 25, pg. 187 (2001)) and possibly other cell types. In addition, MSCs have been shown to provide effective feeder layers for expansion of hematopoietic stem cells (Eaves, et al., Ann. N.Y. Acad. Sci., Vol. 938, pg. 63 (2001); Wagers, et al., Gene Therapy. Vol. 9, pg. 606 (2002)).

Recent studies with a variety of animal models have shown that MSCs may be useful in the repair or regeneration of damaged bone, cartilage, meniscus or myocardial tissues (DeKok, et al., Clin. Oral Implants Res., Vol. 14, pg. 481 (2003)); Wu, et al., Transplantation. Vol. 75, pg. 679 (2003); Mackenzie, et al., Blood Cells Mol. Pis., Vol. 27, pgs. 601-604 (2001)). Several investigators have used MSCs with encouraging results for transplantation in animal disease models including osteogenesis imperfecta (Pereira, et al., Proc. Nat. Acad. Sci., Vol. 95, pg. 1142 (1998)), spinal cord injury (Chopp, et al., Neuroreport. Vol. 11, pg. 3001 (2000); Wu, et al., J. Neurosci. Res., Vol. 72, pg. 393 (2003)) and cardiac disorders (Tomita, et al., Circulation. Vol. 100, pg. 247 (1999). Shake, et al., Ann. Thorac. Sura., Vol. 73, pg. 1919 (2002)). Importantly, promising results also have been reported in clinical trials for osteogenesis imperfecta and enhanced engraftment of heterologous bone marrow transplants.

In addition, in vitro studies from different laboratories have shown that MSCs can inhibit T-cell proliferation either in mixed lymphocyte cultures or by other stimuli such as antigens and mitogens (Di Nicola, et al., Blood, Vol. 99, pgs. 3638-3843 (2002); Tse, et al., Transplantation. Vol. 75, pgs. 389-397 (2003); Aggarwal, et al., Blood, Vol. 105, pgs. 1815-1822 (2005)).

It would be advantageous to provide MSCs that may be employed in the treatment of skin disorders and skin problems, for example, in the treatment of skin burns, skin wounds, skin grafts, scarring, acne, aging from oxidation, skin whitening, wrinkles, skin damage caused by UV rays and skin lifting.

CD146 (cluster of differentiation 146), also known as the melanoma cell adhesion molecule (MCAM) and cell surface glycoprotein MUC18, is a 113 kDa cell adhesion molecule used as a marker for endothelial cell lineage. In humans, the CD146 protein is encoded by the MCAM gene.

### SUMMARY

In one aspect, the present invention provides a composition for treatment of skin disorders and skin problems comprising mesenchymal stem cells wherein said mesenchymal stem cells express CD146 receptors in an amount of at least 10 pg/10⁶ cells.

In another aspect, the present invention provides use of mesenchymal stem cells for the treatment of skin disorders and skin problems, wherein said mesenchymal stem cells express CD146 receptors in an amount of at least 10 pg/10⁶ cells.

In another aspect, the present invention provides a method for treatment of skin disorders and skin problems comprising administration of mesenchymal stem cells wherein said mesenchymal stem cells express CD146 receptors in an amount of at least 10 pg/10⁶ cells.

### BRIEF DESCRIPTION OF THE DRAWINGS

FIG. 1A illustrates the gross appearance of wound scars and wound healing on post-burn day 40, where the burn wounds were treated with a preferred composition of the present invention and controls.

FIG. 1B graphs the results of scar assessment using the Vancouver Scar Scale (VSS).

FIG. 2 illustrates H & E (hematoxylin and eosin) histological sections demonstrating the superior healing of wounds treated with a preferred composition of the present invention compared to controls.

FIG. 3A illustrates the results of Western blot analysis of VEGF in wound sections.

FIG. 3B illustrates the results of Western blot analysis of TGFβ3 in wound sections.

### DETAILED DESCRIPTION OF THE PREFERRED EMBODIMENTS

The present invention relates to MSCs. More particularly, this invention relates to mesenchymal stem cells which express CD146 receptors, and in particular, express CD146 receptors in an amount of at least 10 pg/10⁶ cells.

In accordance with a preferred aspect of the present invention, there is provided a composition comprising mesenchymal stem cells. The MSCs express the CD146 receptors in an amount effective to treat skin disorders and skin problems, for example, in the treatment of skin burns, skin wounds, skin grafts, scarring, acne, aging from oxidation, skin whitening, wrinkles, skin damage caused by UV rays and skin lifting.

In one embodiment, the MSCs express CD146 receptors in an amount of at least 10 pg/10⁶ cells. In another embodiment, the MSCs express CD146 receptors in an amount of at least 12 pg/10⁶ cells, more preferably at least 15 pg/10⁶ cells, and even more preferably at least 18 pg/10⁶ cells.

The inventor has found that MSCs which express CD146 receptors in an amount of at least 10 pg/10⁶ cells are useful in the treatment of skin disorders and skin problems, for example, in the treatment of skin burns, skin wounds, skin grafts, scarring, acne, aging from oxidation, skin whitening, wrinkles, skin damage caused by UV rays and skin lifting. The MSCs of the present invention may work by decreasing the levels of inflammatory factors such as VEGF and TGFβ3, thus having beneficial anti-inflammatory effects.

In one preferred embodiment, the MSCs are obtained from a mammal. The mammal may be a primate, including human and non-human primates. MSCs are isolated from umbilical cord or bone marrow or adipose tissue or any other tissue that contains MSCs. The cells are then sorted into CD146 high. Those CD146 high cells are cultured and expanded to the desired dose. Further details are provided below.

The MSCs may be a homogeneous composition or may be a mixed cell population enriched in MSCs. Homogeneous mesenchymal stem cell compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium.

The amount of cellular CD146 receptors that is expressed in a culture of MSCs may be determined by methods known to those skilled in the art. Such methods include, but are not limited to, quantitative assays such as quantitative ELISA assays, for example. It is to be understood, however, that the scope of the present invention is not to be limited to any particular method for determining the amount of CD146 receptors.

In one embodiment, the amount of CD146 receptors expressed by a culture of MSCs is determined by an ELISA assay. In such an assay, a cell lysate from a culture of MSCs is added to a well of an ELISA plate. The well may be coated with an antibody, either a monoclonal or a polyclonal antibody(ies), against the CD146 receptors. The well then is washed, and then contacted with an antibody, either a monoclonal or a polyclonal antibody(ies), against the CD146 receptors. The antibody is conjugated to an appropriate enzyme, such as horseradish peroxidase, for example. The well then may be incubated, and then is washed after the incubation period. The wells then are contacted with an appropriate substrate, such as one or more chromogens. Chromogens which may be employed include, but are not limited to, hydrogen peroxide and tetramethylbenzidine. After the substrate(s) is (are) added, the well is incubated for an appropriate period of time.

Upon completion of the incubation, a “stop” solution is added to the well in order to stop the reaction of the enzyme with the substrate(s). The optical density (OD) of the sample then is measured. The optical density of the sample is correlated to the optical densities of samples containing known amounts of CD146 receptors in order to determine the amount of CD146 by the culture of MSCs being tested.

Thus, the present invention provides for the selection of a population of MSCs which express CD146 receptors in an amount of at least 10 pg/10⁶ cells. Such selected MSCs then may be admixed with an appropriate pharmaceutical carrier for treatment of the diseases, disorders and problems mentioned hereinabove. For example, the MSCs may be delivered to skin tissue in its own media, in a solution such as a saline solution or Ringer's lactate solution or in a gel. In one preferred embodiment, the MSCs may be administered as a cell suspension including a pharmaceutically acceptable liquid medium for injection.

The MSCs of the present invention are administered to an animal in an amount effective to treat one or more of the above-mentioned diseases, disorders or problems in the animal. The animal may be a mammal, and the mammal may be a primate, including human and non-human primates.

The MSCs may be administered to the skin tissue in a wide variety of ways, for example, by injection, intravenous administration, subcutaneous administration, application of a gel or spray.

The MSCs can be either injected directly to the wound or carried in a matrix gel as part of a composition. Preferably, the composition comprises Integra™, a porous matrix of cross-linked bovine tendon collagen and glycosaminoglycan. The collagen-glycosaminoglycan biodegradable matrix provides a scaffold for cellular invasion and capillary growth. However, any commercial collagen-based or glycose based-matrix may be used in the composition of the present invention.

Alternatively, the MSCs can also be sprayed directly onto the wound area.

The exact dosage of MSCs to be administered is dependent upon a variety of factors, including, but not limited to, the age, weight, and sex of the patient, the disease(s) or disorder(s) being treated, and the extent and severity thereof. The dose can preferably range from about 5,000 MSCs/cm² matrix to 100,000 MSCs/cm² matrix, more preferably about 5,000, 10,000, 20,000 and 40,000 MSCs/cm² matrix.

### Experiments Using Preferred Embodiments

### Materials and Methods

### (1) MSC Preparation

Umbilical cord mesenchymal stromal/stem cells (UCMSCs) were used. MSCs were extracted from the stroma-Wharton's Jelly from umbilical cords. They were cultured in Dulbecco's Modified Eagle's Medium (DMEM), enriched with 1% antibiotic-antimycotic solution, 1% L-Glutamine and 10% fetal bovine serum, and expanded (until cell passage 3-4).

Stem cell differentiation assays were performed to confirm the differentiation potential into the mesenchymal lineages (adipose, cartilage and bone).

Cells were sorted via flow cytometry for MSCs according to the International Society for Cellular Therapy as well as CD146 high. Live cells were selected and gated with the negative markers CD34−/CD11b−/CD45− (FITC), CD19−/HLA−DR− (AF700, PE-Cy7), and positive markers were gated for CD73+ (PE), CD90+ (BV510) and CD105+ (APC) and CD146+ high and CD146+ low.

(2) Cell Incorporation into DRT Integra™

Commercially available DRT Integra™ was used in this preferred embodiment. It has been demonstrated as a reliable cell carrier for tissue engineering which allows cell growth as well as cell differentiation.

First, sorted CD146high UC-MSCs were resuspended and spun down. A cell count for viability was performed.

Second, equal cell distributions for each wound treatment were transferred into 50 ml Falcon tubes containing+25% of cells and 2 ml cell medium (DMEM, enriched with 1% antibioticantimycotic solution, 1% L-glutamine and 10% FBS).

Third, the cells were resuspended and transferred into a petri-dish and homogenously pipetted with a multi-channel-pipette on the acellular Integra™ on top of the bovine collagen, with the silicone side facing down on a sterile cell culture disk.

The cells were seeded on Dermal Regeneration Template (DRT), which builds connections with the wound bed after surgical placement. Each DRT was prepared with 200-2,000,000 cells/cm² according to the experimental protocol.

The porcine MSCs were prepared similarly for control and the acellular control was prepared similarly with a mix of Phospate buffered saline (PBS) and DMEM. Importantly, the DRTs absorbed the entire volume of the cells and PBS suspensions.

Groups were then placed in the incubator at 37° C. at 5% CO₂ until grafting on the pig.

Shortly before surgical grafting, the cellularized scaffolds were assessed under the microscope for floating cells indicating cell death and/or failure to integrate. No floating cells could be detected in any of the scaffolds, indicating full cell integration.

From initial scaffold preparation until surgical grafting, less than 90 min of time had passed. One Integra™ scaffold with a cell density of 5000 cells/cm² was assessed 12 h after cell incorporation and incubation at 37° C. at 5% CO² using a confocal microscope. By imaging, cells were detected until a depth of 123±21 μm in the 1.3 mm thick scaffold, including the silicon bi-layer.

### (3) Full-Thickness Burn Porcine Model

Yorkshire pigs were used (N=3) as they possess similar anatomic and physiologic skin characteristics and comparable pigmentation to humans. Treatment was made to large wound sizes which did not allow spontaneous healing via contracture. The model has been validated from other authors as a sufficient full-thickness burn excised wound model.

One week after being acclimatized and treated with preventive antibiotic for 5 days (ceftiofur injection daily), all three 4-month-old male Yorkshire pigs, with a minimal weight 25 kg and length of 60 cm, were exposed to full thickness burn injuries until the muscle fascia had multiple 5 cm wounds (Total Body Surface Area (TBSA) of 25%) on the dorsal back after a standardized protocol under general anesthesia and analgesia (Buprenorphine 0.05 mg kg-1 subcutaneous, ketamine 0.2 mg kg-1 subcutaneous combined with atropine 0.5-1.0 mg depending on the heart rate, as well as isoflurane 5%/1/O2 intubation).

For wound infliction, a heated aluminum device (200° C.) was used for 20 s with digital force gauge (4.0 N, Mark-10 Corporation) (1N=1 kgms−2) (on day−2). Further analgesia (tramadol 2-4 mg/kg/every 8 h orally) was administered regularly during the experiment. Full thickness burn wounds were histologically confirmed 48-h post-burn via punch-biopsy.

### (4) Wound Treatment

Full-thickness burn tissue excision and hemostasis were performed 48-h post-burn until the muscle fascia on the surgery day (day 0) and wounds were treated with the prepared cellularized DRT, the procine MSCs and the acellular control (Integra™ alone).

The scaffolds were additionally fixed via skin stapler on the wound edges. Regular wound dressing changes (2-3 times/week), as well as 4 mm tissue punch biopsies, were performed at determined time points.

Wound dressing was applied using a layer of topical antibiotics (Polysporin™) fat-gauze (Jelonet™), multiple layers of gauze, as well as adhesive dressing, and a custom-made animal compression jacket.

### (5) Presence of Labeled Cells on the Wounds

Sorted CD146 high UC-MSCs (1,000,000) were labeled with 6 μl of a lipid cell surface dye (eligible for flow cytometry). Additionally, cell viability after labeling was performed according to the manufacturer's protocol and assessed 12 h using Live/Dead™ Viability/Cytotoxicity Kit.

The labeled cells were incorporated with a density of 40,000 cells/cm² into equally cut 5 Å˜5 cm meshed acellular DRT, and were grafted on full thickness burn excised wounds on day 0. Full-thickness tissue biopsies were taken on days 2, 4, 7, and 9 at every dressing change from rotational quadrants of the wounds.

The tissue biopsies were collagenased and analyzed via flow cytometry for detection of a double positive signal with DiO stain on CD90+ cells. Labeled cells (CD90+, DiO) were present in the wound biopsy on the pigs until day 7.

### (6) Wound Healing Assessment

On day 28, photography and biopsies were taken from each wound center and fixed in formalin, followed by 70% EtOH. Paraffin-embedded slides were stained after protocols for Masson's trichrome and immunohistochemistry.

Antibodies used were CD11b, CD163, CD3, and aSMA, which were visualized via HRP polymer detection, followed by betazoid DAB chromogen kits, before mounting and evaluation by light microscopy.

All histology samples were assessed on three different points on the epidermis and in the dermis, measuring in the same depth, from the epidermis 2000 μm into the dermis.

### Results

### (1) Macroscopical Wound Healing

Wound healing was assessed via photography 40 days after treatment, as per the definition in the remodeling phase. The epithelialization area per wound was calculated [(area without epithelialization in cm² on day 40 Å˜100)/initial wound size in cm² on day 0)].

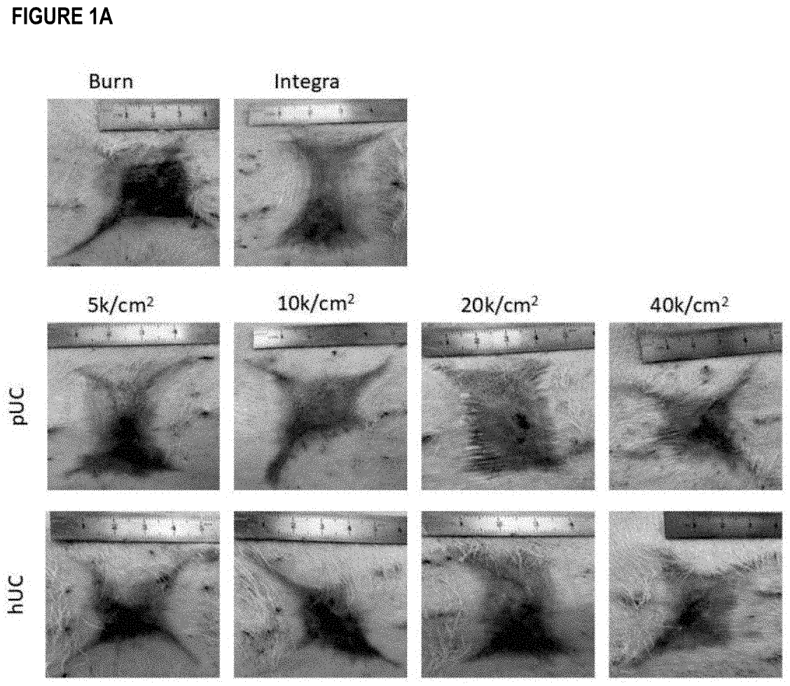

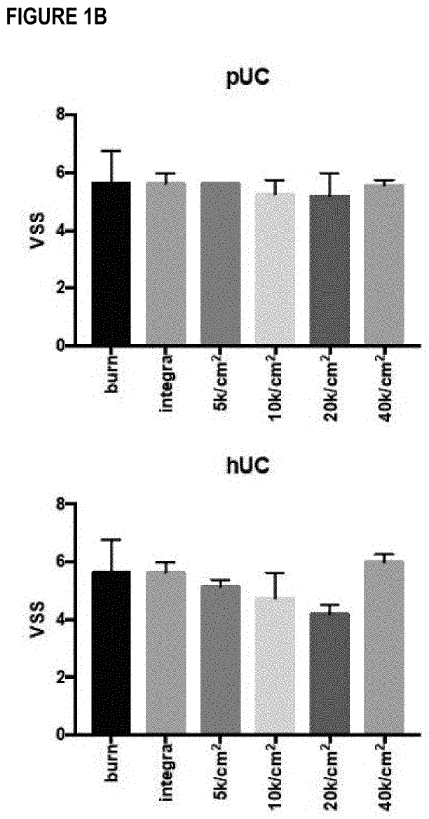

The CD146 high MSC-treated group (hUC) showed a median between 96 and 81% epithelialization compared to the acellular control with a median of 92% (IQR 89-95). The low dose group with 20,000 cells/cm² showed the fastest epithelialization with 96% epithelialization compared to 81% porcine MSCs (pUC) (IQR 91-97), followed by 40,000 cells/cm² with 95% epithelialization (IQR 89-96) (See FIG. 1A).

Scarring was assessed using the Vancouver Scar Scale (VSS, vascularity, pigmentation, pliability, and height), which is the most recognized and validated scar scale.

The CD146 high MSC-treated group of 20,000 cells/cm² showed the lowest scarring with a median VSS of 4 with the narrowest interquartile range (IQR 6-7). The highest dose of 2,000,000 cells/cm² (IQR 4-9) and the lowest dose of 200 cells/cm² (IQR 5-9) both had the same median VSS of 6. The other hMSC-treated groups of 5000, 200,000, and 400,000 cells/cm² showed a median VSS of 6 (all IQR 7-8), compared to the pUC with the same median VSS of 6 (IQR 7-10). Overall the CD146 high MSC-treated groups appeared less inflamed, with a more homogenous scar texture (see FIG. 1B).

### (2) Epidermal Regeneration

Histological assessment was also performed 4 weeks after surgery, where tissue biopsies from the wound centers were taken and stained after Masson's trichrome protocol. For reference, healthy porcine skin representing the physiological condition had a median of 165 μm (IQR 159-182 μm), and burn wounds, without any treatment, had a median of 63 μm (IQR 49-75 μm). Hypo and hyperplasia were defined as inferior or superior epidermal thickness from the interquartile range of the healthy skin.

The best regenerated epidermal thickness was achieved by hUC from the dose of 40,000 cells/cm² with a median of 157 μm (IQR 99-198), followed by the dose of 20,000 cells/cm² with a median of 189 μm (IQR 132-262).

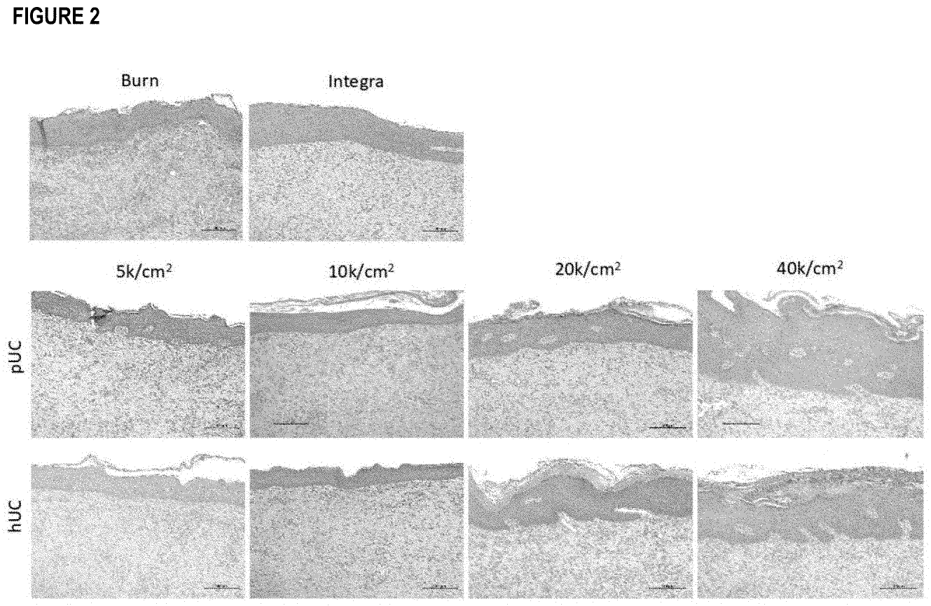

For pUC, the dose of 40,000 cells/cm² showed a median of 131 μm (IQR 116-149). The acellular control showed a median of 177 μm (IQR 64-383 μm), although it lagged in epidermal regeneration and demonstrated a high range of hypo- and hyperplastic epidermal thickness, where the Integra™ scaffold was incompletely degraded by day 28 (See FIG. 2).

### (3) Inflammatory Factors

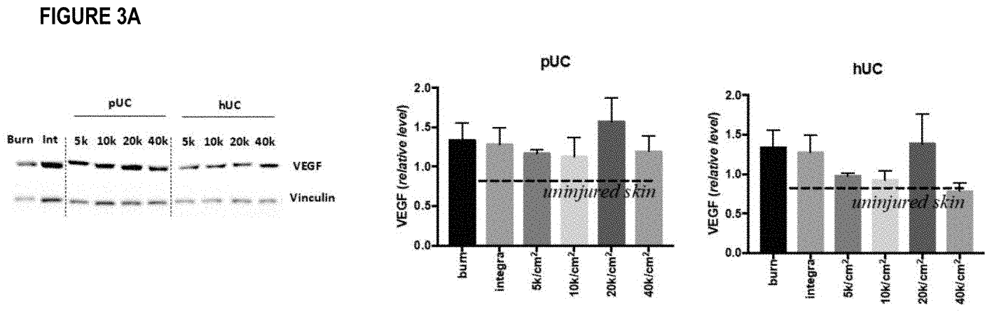

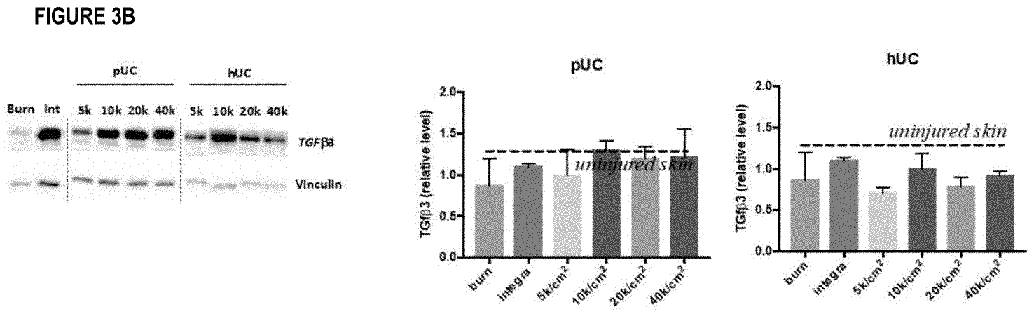

Western blot analysis of VEGF and TGFβ3 showed a significant decrease of TGFβ3 with hUC with lowest levels of 0.65 and 0.75 at 5,000 cells/cm² and 20,000 cells/cm², respectively. In comparison, pUC did not show any significant reduction (See FIG. 3B).

VEGF levels showed decreased levels at 40,000 cells/cm² with hUC, when compared to control, pUC and Integra™ (See FIG. 3A).

### CONCLUSION

These results show that CD146 high Umbilical Cord MSCs (hUC) are significantly better than control and regular porcine Umbilical cord MSCs (pUC) in terms of wound healing, decreased scarring and improving the inflammatory factors at the site of the damaged tissue.

CD146 high Umbilical Cord MSCs (hUC) seem to have better regenerative properties compared to porcine Umbilical cord MSCs (pUC) in regenerative wound healing and potentially other dermatological applications

It is to be understood, however, that the scope of the present invention is not to be limited to the specific embodiments described above. The invention may be practiced other than as particularly described and still be within the scope of the accompanying claims.

What is claimed is:

1. A composition for treatment of skin disorders and skin problems comprising mesenchymal stem cells wherein said mesenchymal stem cells express CD146 receptors in an amount of at least 10 pg/10⁶ cells.

2. The composition of claim 1, wherein said mesenchymal stem cells express CD146 receptors in an amount of at least 12 pg/10⁶ cells.

3. The composition of claim 2, wherein said mesenchymal stem cells express CD146 receptors in an amount of at least 15 pg/10⁶ cells.

4. The composition of claim 3, wherein said mesenchymal stem cells express CD146 receptors in an amount of at least 18 pg/10⁶ cells.

5. The composition of claim 1, wherein said mesenchymal stem cells are human mesenchymal stem cells.

6. The composition of claim 1, further comprising an acceptable pharmaceutical carrier.

7. The composition of claim 6, wherein the composition is in the form of a solution or gel.

8. The composition of claim 7, wherein the pharmaceutical carrier is a saline solution or Ringer's lactate solution.

9. The composition of claim 1, wherein the composition is administered by injection, intravenously, subcutaneously, application of a gel or application of a spray.

10. The composition of claim 1, wherein the skin disorders and skin problems are selected from the group consisting of skin burns, skin wounds, skin grafts, scarring, acne, aging from oxidation, skin whitening, wrinkles, skin damage caused by UV rays and skin lifting.

11. A method for treatment of skin disorders and skin problems comprising administration of mesenchymal stem cells wherein said mesenchymal stem cells express CD146 receptors in an amount of at least 10 pg/10⁶ cells.

12. The method of claim 11, wherein said mesenchymal stem cells express CD146 receptors in an amount of at least 12 pg/10⁶ cells.

13. The method of claim 12, wherein said mesenchymal stem cells express CD146 receptors in an amount of at least 15 pg/10⁶ cells.

14. The method of claim 13, wherein said mesenchymal stem cells express CD146 receptors in an amount of at least 18 pg/10⁶ cells.

15. The method of claim 11, wherein said mesenchymal stem cells are human mesenchymal stem cells.

16. The method of claim 11, further comprising an acceptable pharmaceutical carrier.

17. The method of claim 16, wherein the composition is in the form of a solution or gel.

18. The method of claim 17, wherein the pharmaceutical carrier is a saline solution or Ringer's lactate solution.

19. The method of claim 11, wherein the administration is by injection, intravenously, subcutaneously, application of a gel or application of a spray.

20. The method of claim 11, wherein the skin disorders and skin problems are selected from the group consisting of skin burns, skin wounds, skin grafts, scarring, acne, aging from oxidation, skin whitening, wrinkles, skin damage caused by UV rays and skin lifting.
